(19) United States
(12) Patent Application Publication
(10) Pub. No.: US 2005/0149997 A1
(43) Pub. Date: Jul. 7, 2005

(54) Sterilization of marine organisms by manipulation of DNA content

(76) Inventors: Benjamin L. Wolozin, Newton, MA (US); James M. Sellers, Fort Pierce, FL (US)

Correspondence Address: JACOBSON HOLMAN PLLC, 400 SEVENTH STREET N.W., SUITE 600, WASHINGTON, DC, 20004, US

(21) Appl. No.: 10/980,180

(22) Filed: Nov. 4, 2004

Related U.S. Application Data
(60) Provisional application No. 60/517,199, filed on Nov. 4, 2003.

(51) Int. Cl. A01K 67/033

(52) U.S. Cl. 800/13

(57) ABSTRACT

A novel method of generating a sterile marine organism is disclosed. The method of generating sterile wild type and transgenic marine organisms having triploid genomes is also disclosed. Specifically, we disclose methods of inducing sterility in a number of fish and other species such that their progeny cannot produce offspring by breeding organisms with tetraploid genomes to organisms with diploid genomes to produce triploid offspring.

This application claims priority to U.S. provision patent application No. 60/517,199 filed Nov. 4, 2003 and is hereby incorporated by reference herein as if set forth in the specification in its entirety.

### BACKGROUND OF THE INVENTION

1. Field of Invention

The invention relates to the production of fish and other marine organisms such as crustaceans and mollusks that are incapable of producing offspring. This invention also relates to the methods of producing transgenic fish and other marine organisms such as crustaceans and mollusks that are sterile.

2. Description of Prior Art

Aquaculture is a major industry throughout the world. Increasingly, aquaculture professionals, the public, public interest groups and governmental agencies are concerned with preventing uncontrolled growth of exotic fish species in the wild due to accidental or intentional release of these fish. For instance, aquaculture groups developing transgenic fish, such as ornamental fish expressing transgenic fluorescent proteins or rapidly growing salmon expressing transgenic growth hormone want to prevent both the growth of these fish in the wild following accidental release, and the unauthorized farming of the fish following commercial purchases. Furthermore, environmental groups and governmental agencies want to prevent contamination of wild salmon stocks by farmed salmon or similar aquatic organisms that are released accidentally and then breed. The importance of these issues is emphasized by recent regulations instituted in California that require registration of all transgenic marine organisms that are grown in the state for commercial use. These examples demonstrate that there is extensive concern about uncontrolled growth of exotic fish and other marine organisms in the wild.

Two solutions to the problem of introducing transgenic fish accidentally into the wild have been proposed. The first solution is containment. However, containment is not perfect and provides no protection from uncontrolled release should it fail and accidental release of exotic fish occurs. A second approach is to induce triploidy in fish. Triploid fish are viable, but sterile. Sterilizing fish is an attractive approach to preventing uncontrolled growth of exotic fish or other marine organisms following release because it insures that any released organisms will be unable to breed.

Previous literature demonstrates that triploidy can be induced in fish by exposing fish eggs to a stress approximately 20 minutes after fertilization. F. M. O'Flynn et al., Experiments On The Use Of Triploidy To Produce Sterility In Atlantic Salmon (The Atlantic Salmon Federation) (http://www.asf.ca/Research/resrch09.htm); Yang H., et al. Triploid and Tetraploid Zhikong Scallop, Chlamys farreri Jones et Preston, Produced by Inhibiting Polar Body I., Mar Biotechnol (NY). 2000 September; 2(5): 466-475; Felip A., et al., Induction of triploidy and gynogenesis in teleost fish with emphasis on marine species, Genetica 2001; 111(1-3): 175-95; Eudeline B., et al, Delayed meiosis and polar body release in eggs of triploid Pacific oysters, Crassostrea gigas, in relation to tetraploid production, J. Exp. Mar. Biol. Ecol 2000 May 31; 248(2): 151-161; Felip A., et al., The relationship between the effects of UV light and thermal shock on gametes and the viability of early developmental stages in a marine teleost fish, the sea bass (Dicentrarchus labrax L.), Heredity. 1999 October; 83 (Pt 4): 387-97; Lamatsch D K, et al., Unusual triploid males in a microchromosome-carrying clone of the Amazon molly, Poecilia Formosa, Cytogenet Cell Genet. 2000; 91(1-4): 148-56; Herbst, Eric C. Induction of Tetraploidy in Zebrafish Danio rerio and Nile Tilapia Oreochromis niloticus, Master's Thesis, Jun. 5, 2002 (Louisiana State University http://etd01.lnx390.lsu.edu:8085/docs/available/etd-0711102-135713/unrestricted/) The types of stresses that have been examined to date are heat shock, cold shock, chemical shock and pressure shock. The efficacy of the particular method for inducing triploidy (e.g., heat or cold) varies depending on the fish. However, the utility of triploidy as a method for sterilizing fish suffers because the induction of triploidy is frequently incomplete, in that only a fraction of the offspring become triploid following any given stress. This means that there is always a risk that some of the fish treated to induce triploidy will not be triploid, and these fish will be fertile. Because of incomplete induction of triploidy following physical stresses, there is clearly a need to develop a method for inducing triploidy that is more secure.

We have found that we can induce genome duplication by taking the fertilized eggs of a fish and timing a shock to the eggs so that the embryo develops as a tetraploid instead of a diploid. The methods for inducing genome duplication (octaploidy in Zebrafish (Danio rerio); tetraploidy in Rosy Barb (Barbus conchonius)) involve fertilizing the clutch of eggs via in vitro fertilization and administering a heat shock to the clutch at a predetermined time post-fertilization, and for a predetermined duration, and at an optimum temperature that is determined by the method described below. Our findings are somewhat similar to a related report, by Herbst as part of a thesis, however Herbst incorrectly characterizes the ploidy of the Zebrafish and does not work for other fish, such as Rosy Barb (Herbst, 2002).

### SUMMARY OF THE INVENTION

It is an object of the invention to develop fish and other marine organisms that are uniformly triploid and uniformly sterile. It is another object of the invention to develop sterile transgenic fish species and other marine organisms including but not limited to: False Percula; Clownfish—Amphiprion ocellaris; Gold-Stripe Maroon Clownfish—Premnas biaculeatus; Saddleback Clownfish—Amphiprion polymnus; Australian Clownfish—Amphiprion rubrocinctus; Orchid Dottyback—Pseudochromis fridmani; Striped Dottyback—Pseudochromis sankeyi; Blue-Striped Dottyback—Pseudochromis springeri; Salmon (all varieties); Tilapia; and Trout (all varieties).

We have recently determined the conditions for duplicating the Zebrafish genome, using fluorescent activated cell sorting to analyze the DNA content and determine the precise conditions for inducing tetraploidy. In addition, we have analyzed the genomes of other fish, such as Rosy Barbs; Cherry Barbs; Neon Tetras; Salmon; False Percula Clownfish—Amphiprion ocellaris; Gold-Stripe Maroon Clownfish—Premnas biaculeatus; Saddleback Clownfish—Amphiprion polymnus; Australian Clownfish—Amphiprion rubrocinctus; Orchid Dottyback—Pseudochromis fridmani; Striped Dottyback—Pseudochromis sankeyi; and Blue-Striped Dottyback—Pseudochromis springeri to determine the ploidy of their genomes. In the process we determined that some fish, such as Rosy Barbs have diploid genomes, while other fish have tetraploid genomes, such as Zebrafish. Using this knowledge, we then determined conditions that would allow generation of tetraploid Rosy Barbs and other marine organisms. In addition, we have shown that the water conditions reported for Zebrafish are inadequate to support the viability of Rosy Barbs and some other species following this procedure, and have developed methods for enabling fertilized, heat shocked Barbs and other species of aquatic life to be viable.

We propose a novel method for inducing triploid fish and marine organisms by breeding diploid fish with tetraploid fish or by fertilizing tetraploid germ cells from a male or female with diploid germ cells from a fish of the opposite sex. The resulting offspring are triploid and will be sterile. Diploid organisms, such as Rosy Barbs, are fertile and viable. By fertilizing eggs from a diploid female with sperm from a tetraploid male, or by fertilizing eggs from a tetraploid female with sperm from a diploid male we propose to generate a population of offspring that is uniformly triploid and uniformly sterile. The reason that this occurs is because tetraploid animals generate germ cells following meiosis that have a 2n complement of DNA. Similarly diploid animals generate germ cells following meiosis that have a in complement of DNA. When the in germ cell interacts with the 2n germ cell, the result is a 3n (triploid) fertilized egg that will grow up to produce a 3n (triploid) sterile adult. We have also shown that tetraploid Rosy Barbs can be grown past the larval stage and are viable.

We also propose a similar embodiment of the method for inducing hexaploid fish (5n) and marine organisms by breeding tetraploid fish with hexaploid fish or by fertilizing tetraploid germ cells from a male or female with octaploid germ cells from a fish of the opposite sex. Such hexaploid offspring are also sterile.

### BRIEF DESCRIPTION OF THE DRAWINGS

FIG. 1: FACS data showing a peak corresponding to normal diploid (2n) Zebrafish where peak level control value is 200 instead of 100 for Rosy Barbs.

FIG. 2: Diagram showing thermocycler protocols for Danio rerio and Barbus conchonius.

FIG. 3: FACS data showing a peak corresponding to normal tetraploid (4n) Zebrafish at control temperature.

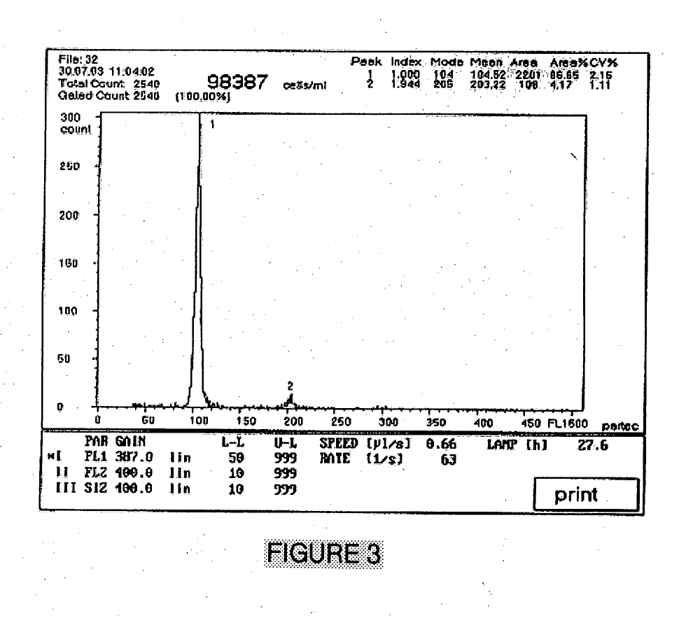

FIG. 4: FACS data showing a peak corresponding to normal tetraploid (2n) Zebrafish after incubation at 41° C.

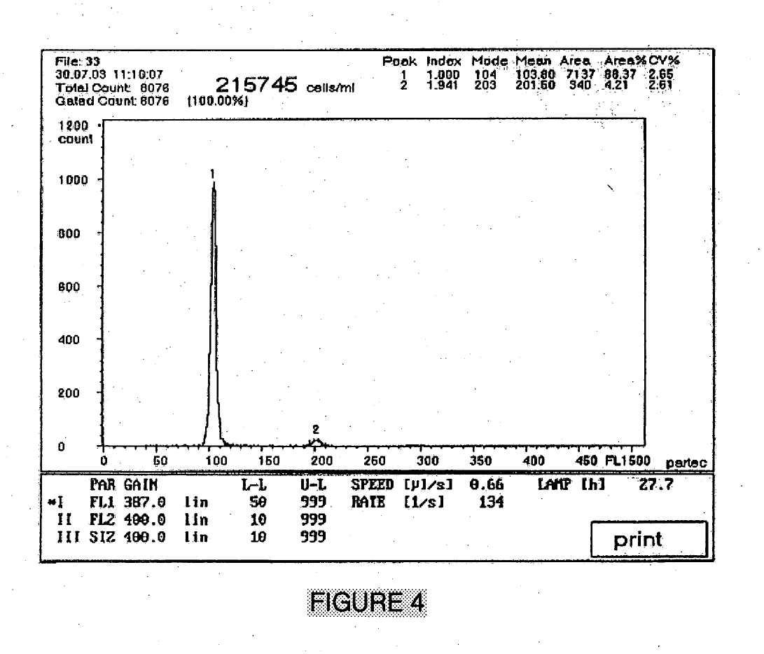

FIG. 5: FACS data showing a peak corresponding to octaploid (4n) Zebrafish after incubation at 41° C.

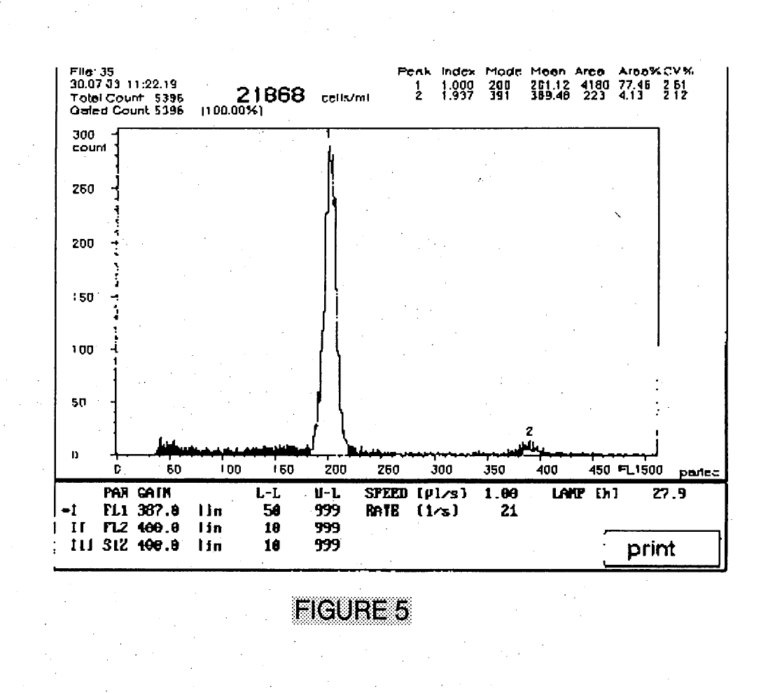

FIG. 6: FACS data showing a peak corresponding to normal diploid (2n) Rosy Barbs.

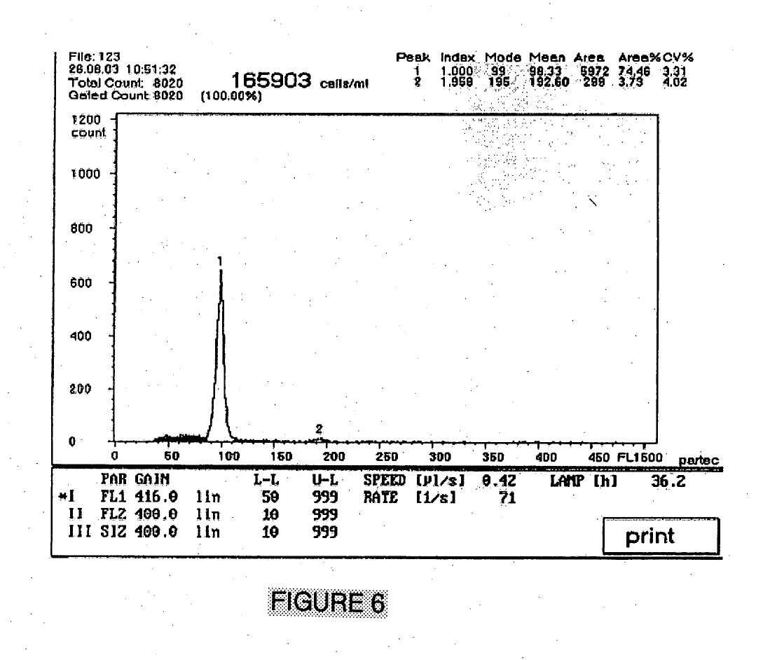

FIG. 7: FACS data showing a peak corresponding to normal diploid (2n) Rosy Barbs after incubation at 40° C.

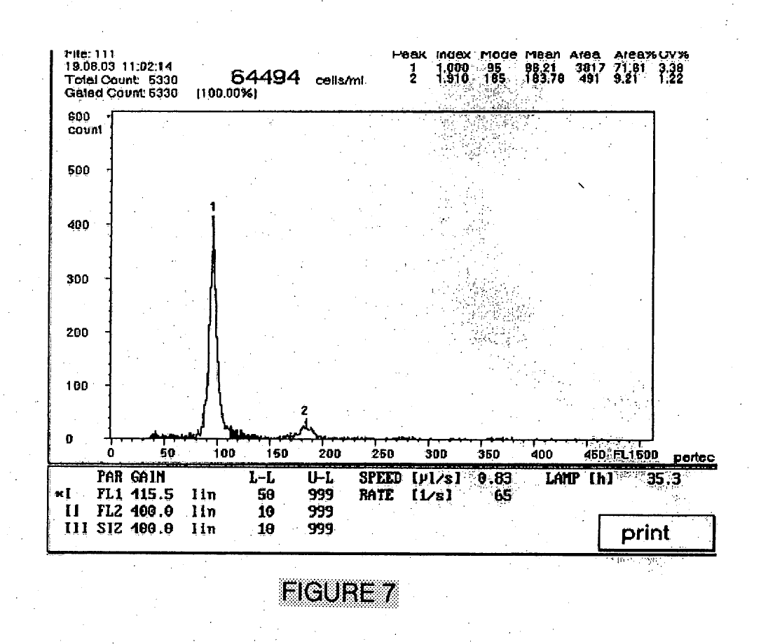

FIG. 8: FACS data showing a peak corresponding to tetraploid (4n) Rosy Barbs after incubation at 40.6° C.

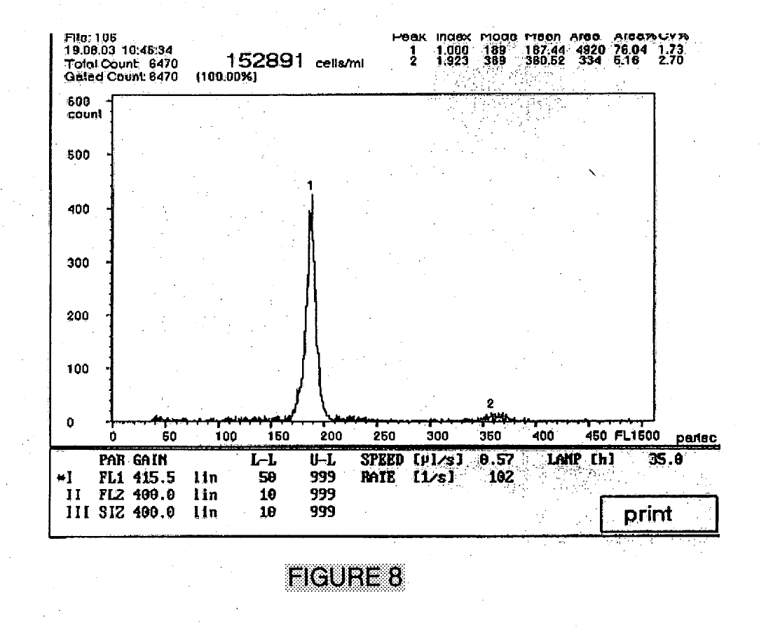

FIG. 9: FACS data showing a peak corresponding to normal diploid (2n) Rosy Barbs adult fin clip.

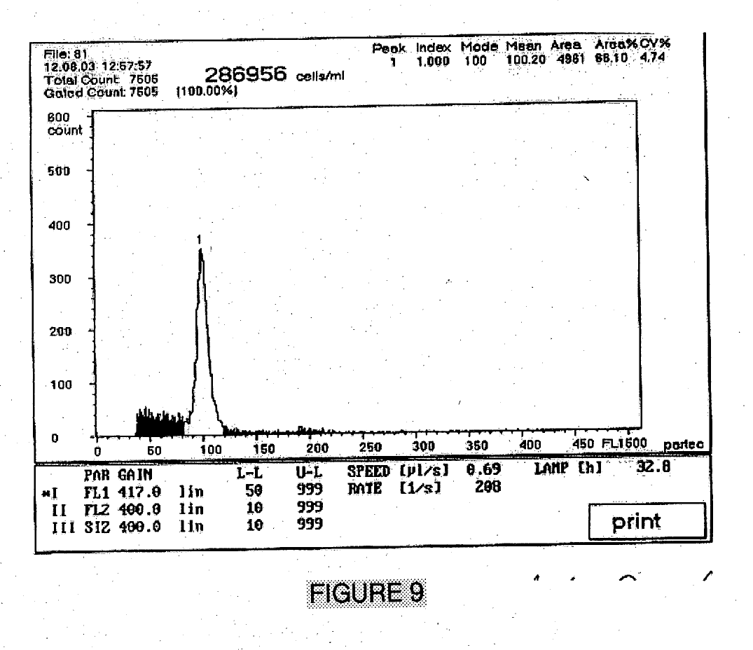

FIG. 10: A graph showing generation of genome duplicated (8n) Zebrafish is temperature dependant. The X-axis shows the incubation temperature in degrees C., and the Y-axis shows the number of larvae. The dots represent to the number of larvae exposed the temperature, the squares represent to the number of larvae surviving the process, and the triangles represent the number of tetraploid larvae. The small dashed lines represent (2 per. Mov. Avg. (#4n), the large dashed lines represent (2 per. Mov. Avg. (# survived), and the solid line represents (2 per. Mov. Avg. (#exposed).

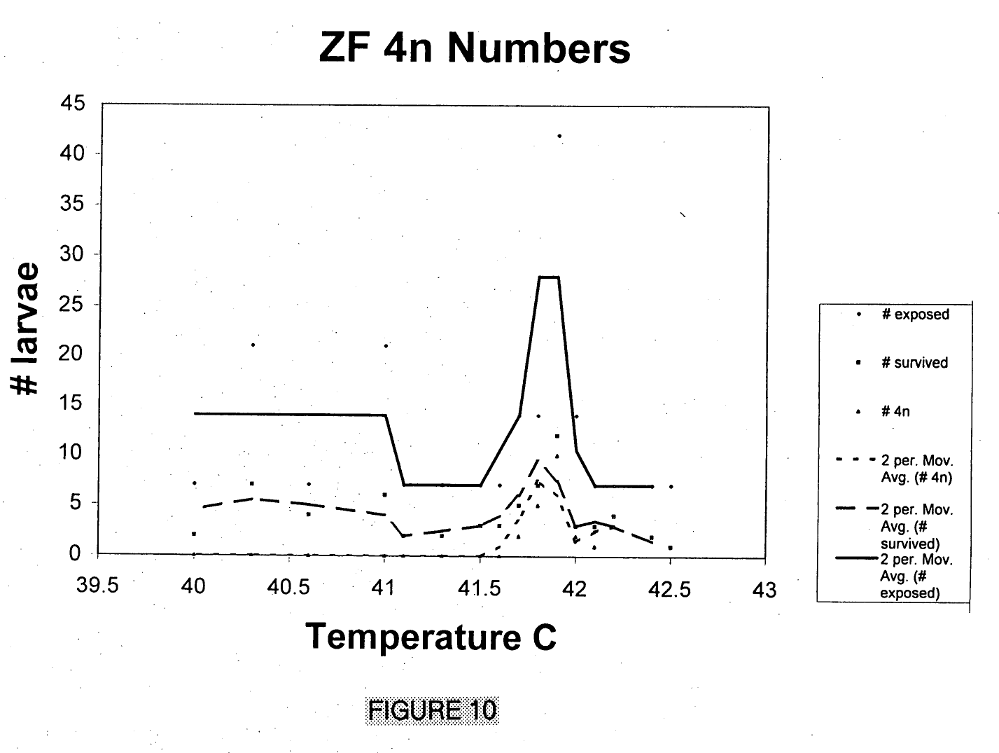

FIG. 11: A graph showing the temperature dependant generation of genome duplicated (8n, octaploid) Zebrafish and the percent survival of the genome duplicated larvae. The line with the square points represents the percentage of larvae that became genome duplicated after incubation at that temperature, and the line with the diamonds represents the number of genome duplicated larvae that survived at that temperature.

FIG. 12: A graph showing the temperature dependant generation and survival of tetraploid (genome duplicated) Rosy Barbs.

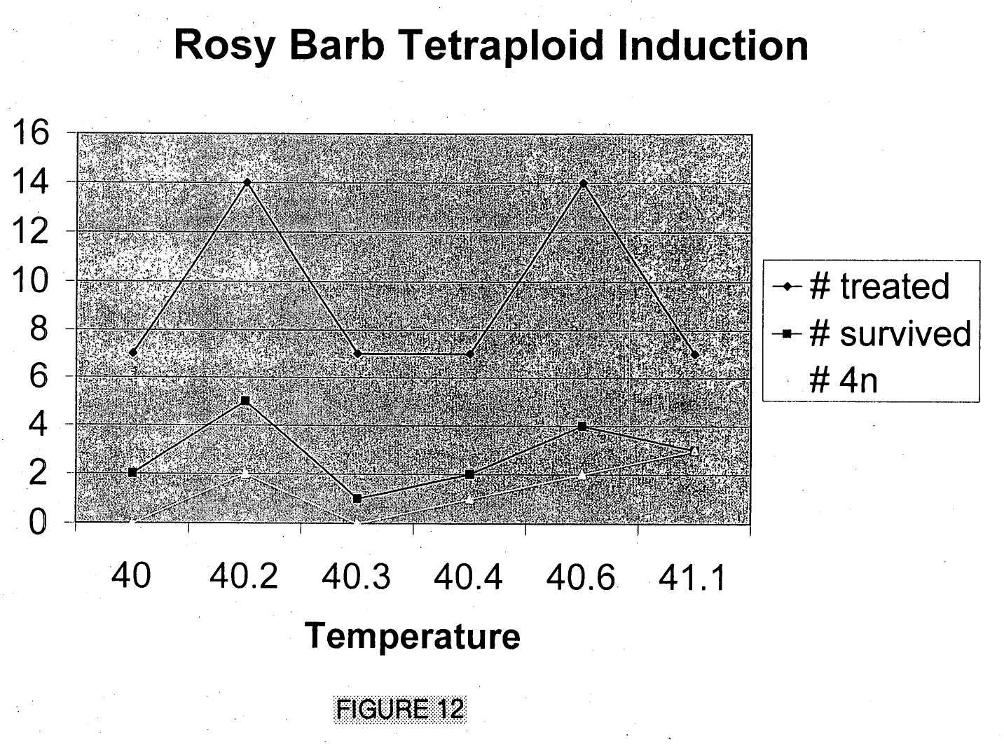

FIG. 13: FACS data showing a peak corresponding to normal diploid (2n) Rosy Barbs where peak level control value is 100.

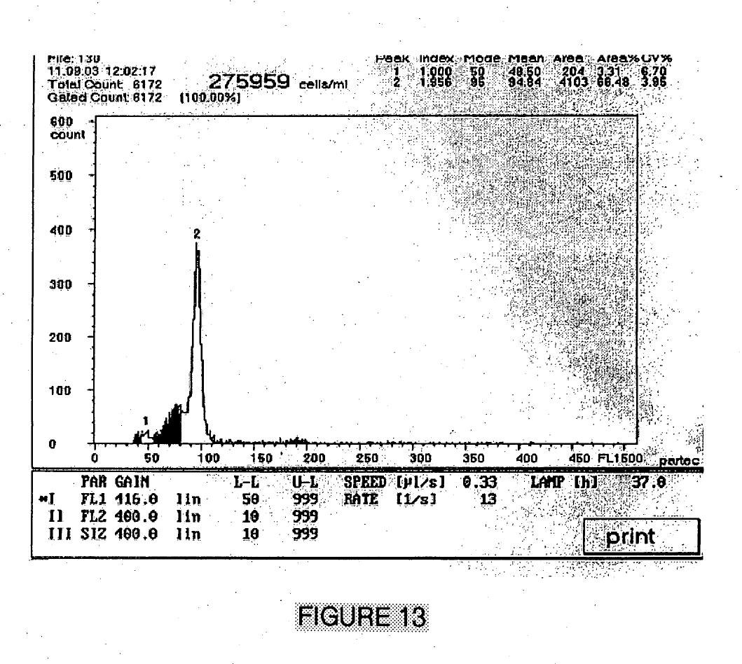

FIG. 14: FACS data showing a peak corresponding to diploid (2n) Amphiprion ocellaris.

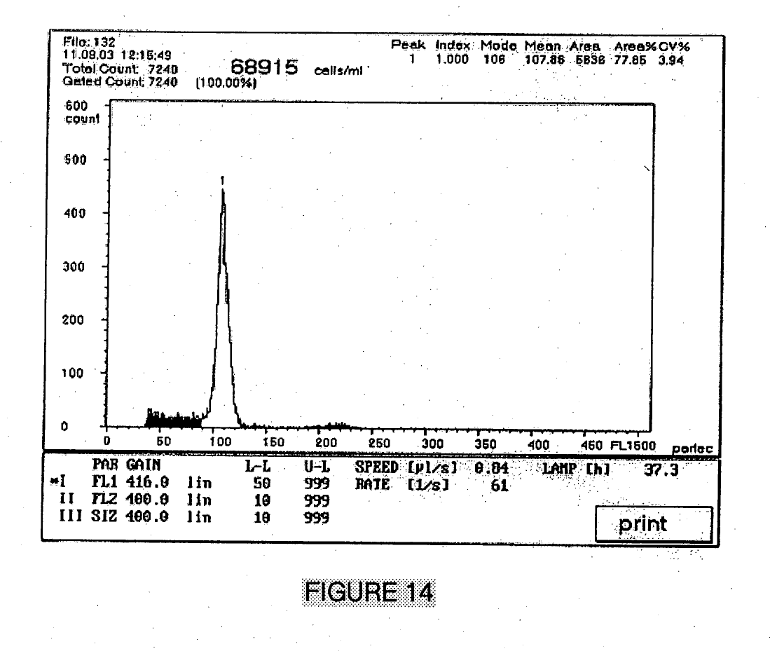

FIG. 15: FACS data showing a peak corresponding to diploid (2n) Premnas biaculeatus.

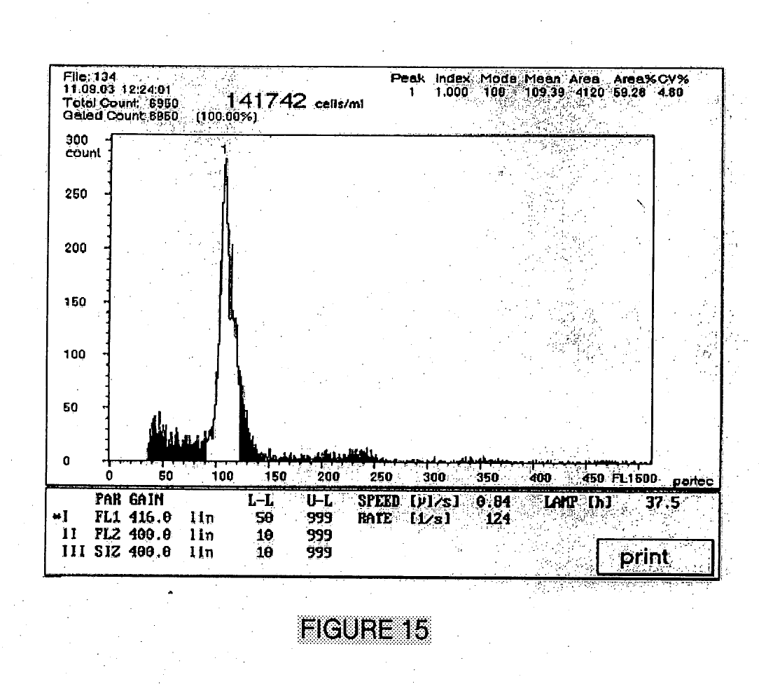

FIG. 16: FACS data showing a peak corresponding to diploid (2n) Amphiprion polymnus.

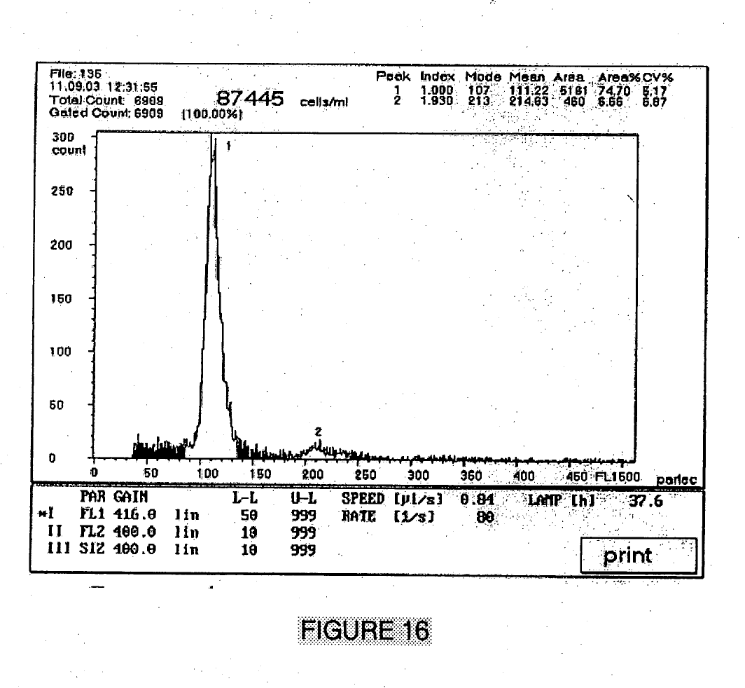

FIG. 17: FACS data showing a peak corresponding to diploid (2n) Amphiprion rubrocinctus.

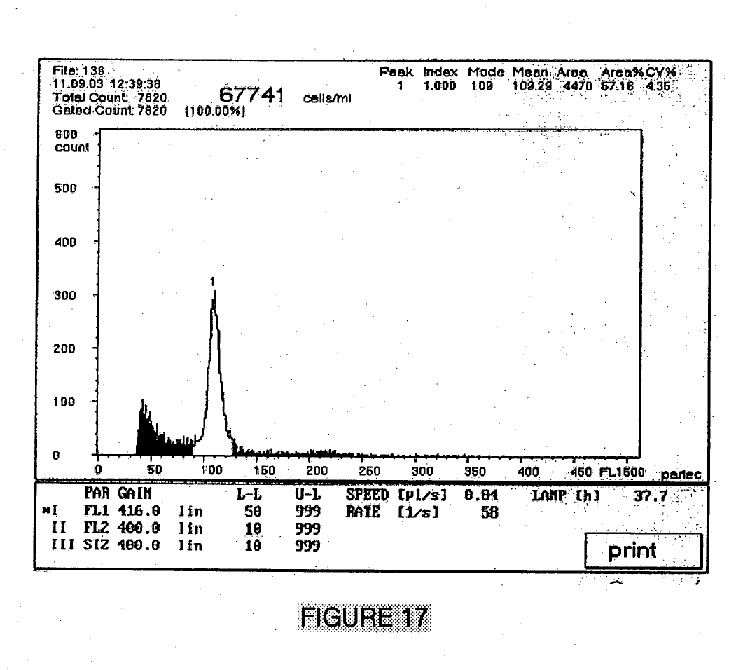

FIG. 18: FACS data showing a peak corresponding to diploid (2n) Pseudochromis fridmani.

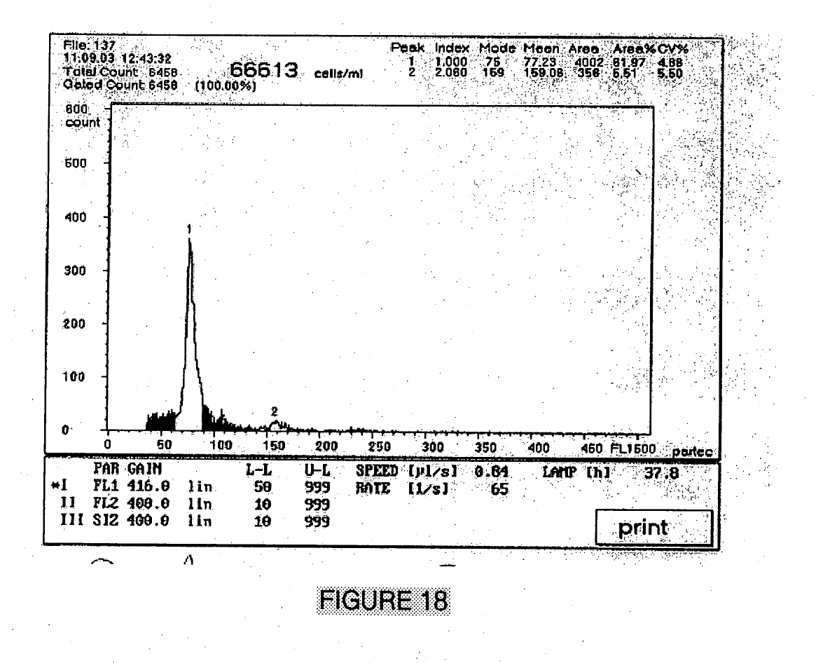

FIG. 19: FACS data showing a peak corresponding to diploid (2n) Pseudochromis sankeyi.

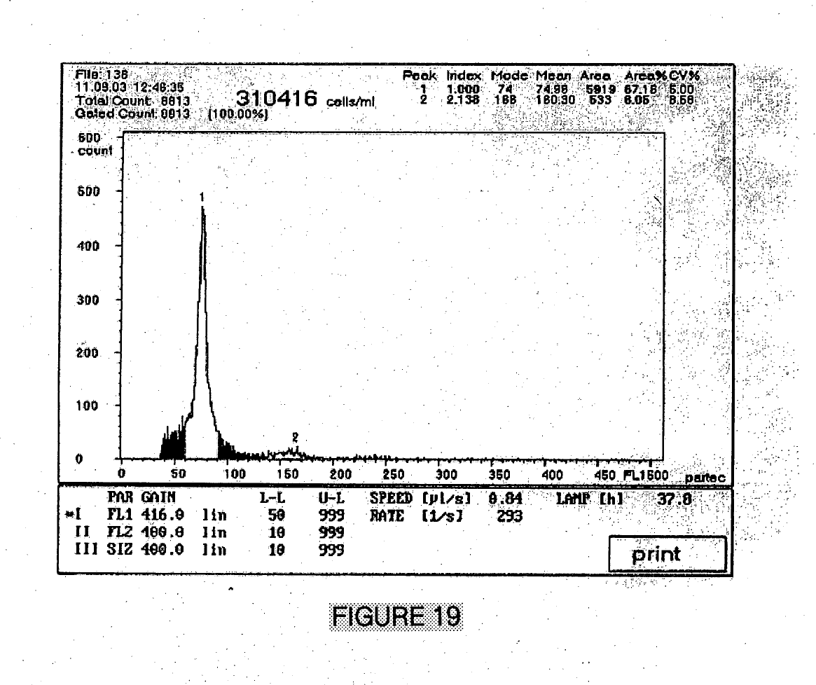

FIG. 20: FACS data showing a peak corresponding to diploid (2n) Pseudochromis springeri.

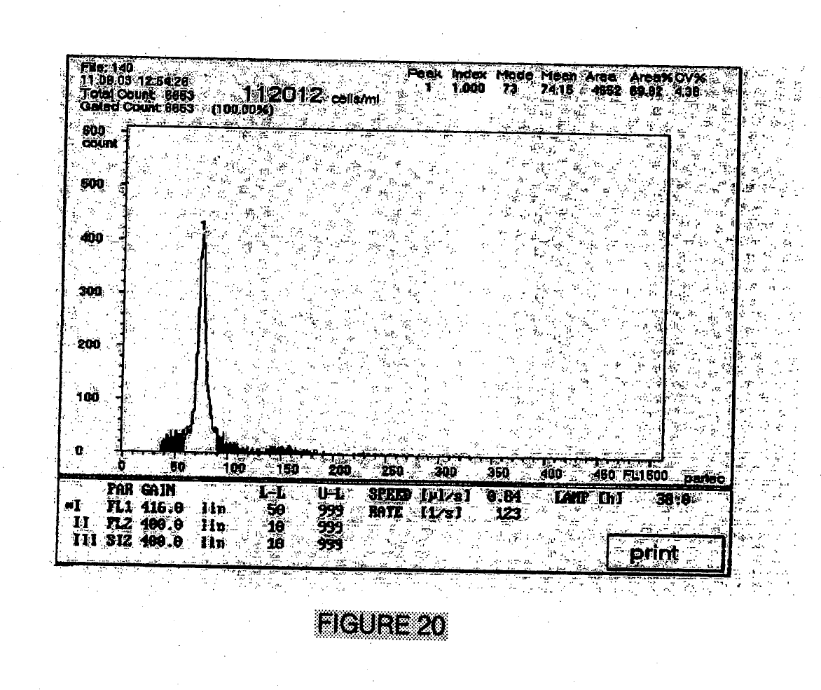

### DETAILED DESCRIPTION AND PREFERRED EMBODIMENTS

Zebrafish have been the subject of a number of publications describing insertions of transgenic constructs. See, Higashijima S. et al., High-frequency generation of transgenic Zebrafish which reliably express GFP in whole muscles or the whole body by using promoters of Zebrafish origin, Dev Biol. 1997 Dec. 15; 192(2): 289-99; The Aequorea victoria green fluorescent protein can be used as a reporter in live Zebrafish embryos, Dev Biol. 1995 September; 171(1): 123-9; Amsterdam A., et al., Requirements for green fluorescent protein detection in transgenic Zebrafish embryos, Gene 1996; 173(1 Spec No): 99-103; Long Q., et al., GATA-1 expression pattern can be recapitulated in living transgenic Zebrafish using GFP reporter gene, Development. 1997 October; 124(20): 4105-11; Gibbs P D, et al., An in vivo screen for the luciferase transgene in Zebrafish, Mol. Mar. Biol. Biotechnol. 1994 December; 3(6): 307-16; Gibbs P D, et al., Inheritance of P element and reporter gene sequences in Zebrafish, Mol. Mar. Biol. Biotechnol. 1994 December; 3(6): 317-26; and U.S. Pat. No. 6,380,458, entitled, “Cell-lineage Specific Expression in Transgenic Zebrafish”, issued Apr. 30, 2002 to Shuo Lin and assigned to the Medical College of Georgia Research Institute, Inc.

A detailed description of a general method of making transgenic fish, and specifically, transgenic Zebrafish (Brachydanio rerio) can be found in Higashijima S. et al., Amsterdam, A. et al, and Gibbs, P D et al references, as well as in PCT Application WO 00/49150, by Gong, Z. et al., entitled, “Chimeric Gene Constructs for Generation of Transgenic Ornamental Fish”. All references, including publications, patents and patent applications disclosed within are hereby incorporated by reference in their entirety. A general discussion of creation of transgenic fish follows.

The production of transgenic fish and other marine organisms has several advantages over methods employing other transgenic hosts (Gong and Hew, Curr. Topics Dev. Biol., 30: 177-214 (1995)). One individual fish provides from 100's to 1000's of eggs, so that the availability of eggs for DNA transfer is not limiting. Fertilization is external, allowing easy collection of freshly fertilized eggs. Some species of fish, such as tilapia, produce eggs every 2-3 weeks, allowing large numbers of fish and gametes to be produced in a very short period of time, DNA can be introduced into the fertilized eggs by any conventional technique for introduction of DNA into a cell such as the preferred method of microinjection, the DNA gun, transfection or electroporation. Another useful method is electroporation, a technique in which the eggs are bathed in a solution of the DNA to be incorporated and an electric current is applied which opens pores in the eggs long enough for surrounding DNA to enter the egg. The use of this technique requires no special skill on the part of the investigator, and hundreds of potential transgenic fish can be created within a single experiment (Inoue et al., Cell. Differ. Dev., 29: 123-129 (1990)). After fertilization, fish develop without further manipulation and can be tested for the presence of a transgene within a week after hatching, using standard methods. The short generation time for several species of fish also offers an advantage over large mammalian species.

Expression of Genes in Fish

In order to express a recombinant protein from DNA in a living system, it is necessary to include sequences that direct the transcription of the gene, the gene sequence encoding the protein, and sequences that direct the termination of transcription of the gene. Sequences that direct the transcription of a gene are generally regions located adjacent to the 5′ end of a gene and are termed “promoters”. Transcription termination signals are located beyond the 3′ end of the coding region of the gene and often contain sequences AATAAA followed by stretches of variable length comprising pyrimidine rich sequences. Genes to be expressed may be derived from cDNAs produced from mRNA isolated from any biological source expressing the gene of interest, or alternatively, may be derived from genomic DNA isolated from the species of interest. Genomic DNAs are often very large, containing not only the sequence for the gene, but a variable number of introns (intervening sequences removed from the RNA transcribed from a gene) as well. There may be some advantage to including introns in genes to be expressed in transgenic animals.

Many promoters have been used in attempts to produce transgenic fish with economically desired characteristics, including those isolated from mouse leukemia virus (MoMLV), mouse κ-immunoglobulin gene, SV40 virus, promoters containing the Rous sarcoma virus long terminal repeat (RSV LTR), mouse metallothienin gene, Xenopus laevis elongation factor 1α-gene, carp β-actin gene, human heat shock protein gene, ocean pout antifreeze protein gene and the VSV promoter (Fletcher and Davies, Genetic Engineering 13: 331-370 (1991); Liu et al., Bio/Technology 8: 1268-1272 (1990); Du et al., Biotechnology 10: 176-181 (1992)). All have yielded limited expression in species of fish studied (goldfish, loach carp, rainbow trout, Atlantic salmon); few except carp β-actin promoter, ocean pout antifreeze promoter, the myosin promoter, the keratin promoter, the heat shock promoter, as well as some very obscure promoters are native to fish. It is expected that promoter regions isolated from fish will be optimally expressed in a fish system, since the regulatory regions are expected to optimally bind to regulatory proteins residing in the homologous species.

### EXAMPLE 1

### Brachydanio rerio

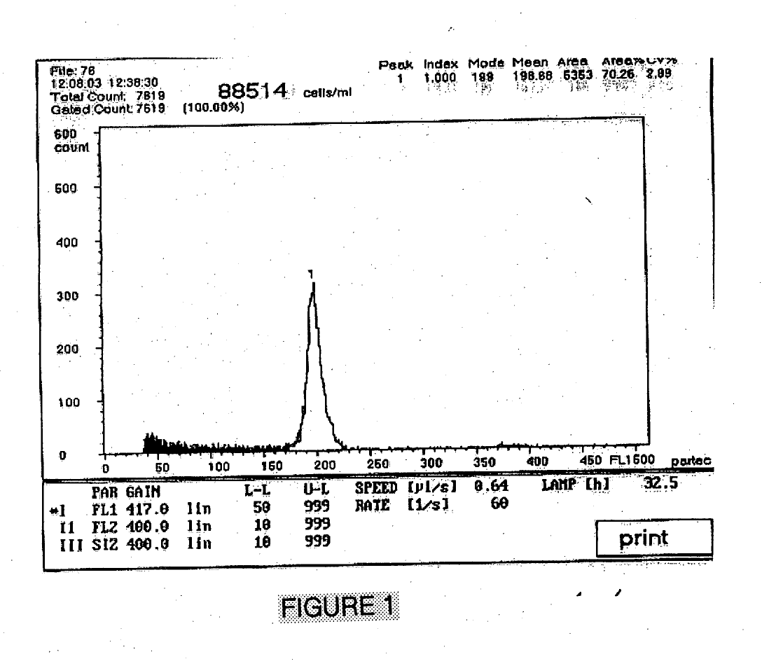

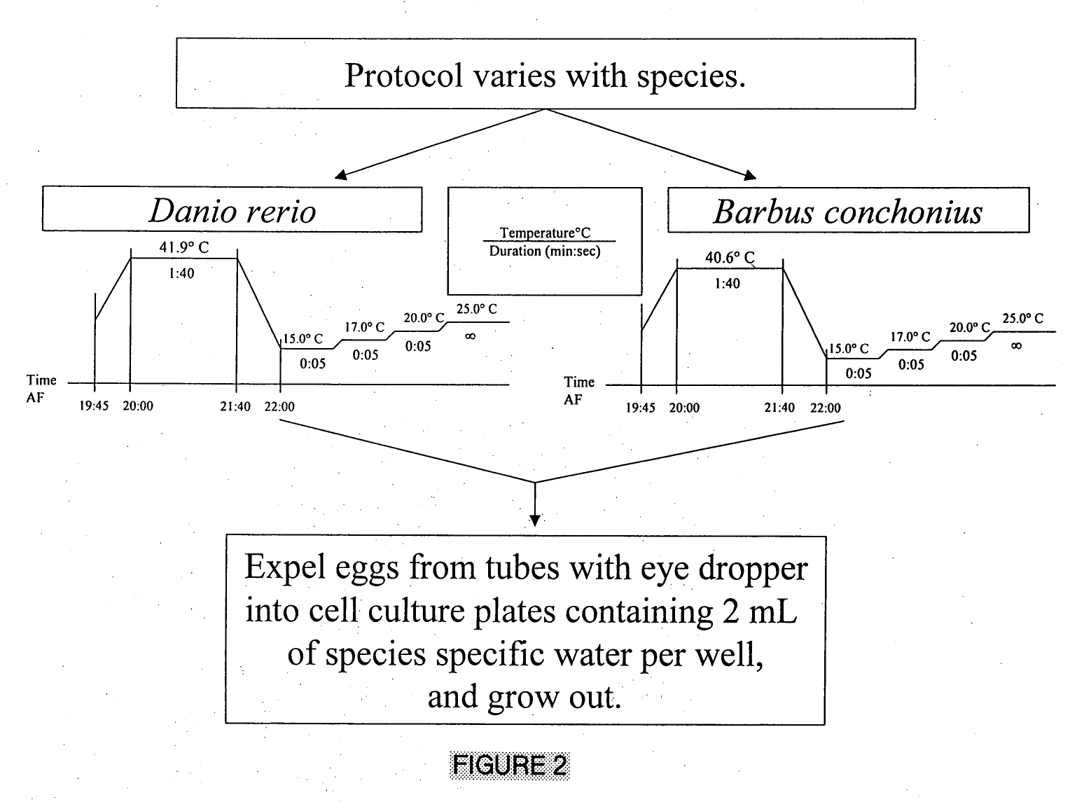

Tetraploid induction begins by taking the fertilized eggs of a fish and timing a shock to the eggs so that the embryo develops as a tetraploid instead of a diploid. FIG. 1 shows data collected from a fin clipping of normal diploid (2n) Zebrafish using a fluorescence activated cell sorter (FACS). The method for inducing genome duplication (octoploidy in Zebrafish (Danio rerio)) involves fertilizing a clutch of eggs via in vitro fertilization and administering a heat shock to the clutch precisely twenty minutes post fertilization, and for a duration of about two minutes at a temperature of about 41.3° C. to about 42.0° C. The heat shock does not have to be exactly two minutes, and somewhat shorter durations of about 1 min, 45 sec or longer (e.g., 2m, 15 sec) also suffice. FIG. 2 gives a schematic illustration of the temperature protocol. It is important to note that the approximate temperature ranges and time ranges given for the protocols discussed throughout this specification are approximate, and can vary by at least 10 to 20%.

In a small pipette tip box, set up twenty-four 0.2 mL microcentrifuge tubes leaving the lids open. Pipette 100 μL of distilled water into each tube in preparation for shocking Danio rerio. The temperature protocols for shocking the fish using the BIO-RAD iCycler are as follows: The protocol for Danio rerio begins with a 1:40 (min:sec) interval about 41.3° C. to about 42.0° C. This allows for a twenty-second ramp down in temperature to complete a two-minute heat shock. The next intervals are 15° C. for 0:05, 17° C. for 0:05, 20° C. for 0:05 and finally a 25° C. hold, at the end of the cycle.

In Vitro Fertilization Method: Eggs and sperm are obtained from adult fish (both wild type fish and fish with duplicated genomes in the following manner. The males are anesthetized by incubation in a 4% solution of Tricaine (or any solution sufficient to temporarily paralyze the fish to allow harvesting of eggs) until the fish become immobile. The anesthetized males then have sperm stripped from them using a 10 μL pipette tip, and the sperm is then placed on ice. The males are then resuscitated and then the females are anesthetized in the same manner. The eggs are stripped from the females by digital manipulation and then placed into 100 mm×15 mm petri dishes (Fisher #08-757-12). The females are then resuscitated.

The healthiest looking clutch and sperm samples are then selected to use for the fertilization. Healthy clutches are those that by visual inspection have yellowish in color, with no white eggs (dead eggs), and having a consistency thicker than water. The selected sperm and clutch samples are then mixed. About 2 to 3 mL of distilled water are added to the clutch and mixed. The addition of water activates the sperm and induces fertilization, and is carefully timed using a stopwatch. Fill a petri dish with approximately 15 mL with distilled water and take the dish to load into the microcentrifuge tubes.

Aspirate approximately 100 eggs with an eyedropper or pipette having a large enough opening so that it will not harm the eggs (i.e., the opening must be wide enough to take up the eggs without inducing trauma to the eggs), and dispense them onto a standard microscope slide. Using a Pasteur pipette, place the tip of the pipette so that it is in the water/egg mixture and against the slide. Aspirate the water with the Pasteur pipette, leaving the clutch of eggs on the slide. Using a moistened, small, fine tip brush (similar to a nail polish brush) manipulate the eggs into groups of seven, sweep the groups off of the slide and place them into a microcentrifuge tube (any brush or utensil capable of transferring the eggs from the slide to the microcentrifuge tube will suffice). Once all 24 tubes are loaded with eggs, cap the tubes and place them into the thermocycler. See diagram in FIG. 2.

With the thermocycler loaded, begin the desired protocol making sure to enter the appropriate volume (100 μL) into the thermocycler's pre-run page. Next begin the thermocycler protocol, and then pause it immediately to wait for the timer to reach 19 min 45 sec after fertilization. Once this time has been reached, resume the run. Once the protocol has run all the way through and is holding at about 25° C., unload the tubes and put them back into the pipette tip box. Open the 24 well culture cluster plate (Corning, Inc. item 3524) and obtain at least 200 mL of distilled water and an eyedropper. Open each tube, invert it, and squirt the water up into the tube using the eyedropper to expel the embryos into a well in the 24 well culture cluster plate. Once all 24 wells are filled, incubate at approximately 28.5° C. Grow out the embryos and evaluate ploidy by doing FACS analysis on their fin clips.

The above target temperatures were reached using the gradient feature on the thermocycler. The temperatures for inducing genome duplication were chosen using iterative studies with increasingly narrow temperature gradients to optimize the temperatures for induction of genome duplication, and balancing survival with expression. In vitro fertilization is then performed either with sperm from a wild type male and a genome duplicated female, or with sperm from a genome duplicated male and a wild type female. The fertilized eggs are then grown up and will be sterile.

### EXAMPLE 2

### Barbus conchonius

Conditions used and documented as successful for the rearing of microinjected Zebrafish (Danio rerio) larvae proved to be inadequate when transferred across species to Rosy Barbs. Survival rates of zero were frequently observed in this situation. Therefore, research into the difference of the two species yielded a suggestion to change the water quality parameters in two distinct manners. The adult Rosy Barb broodstock is kept in aqueous medium at a pH of 6.0 while the Zebrafish are kept at pH 7.0. Also, the Rosy Barbs are far more productive when organic material is added to the aqueous medium, whereas Zebrafish do not require this to be prolific. One method for adding organic material is to soak the aqueous medium in peat. Another method is to use an organic liquid concentrate that is added to the broodstock aqueous medium. The latter method is what was used with the Rosy Barbs here at PiPets. So those two parameters were changed to be more appropriate for the Rosy Barb embryos. Also, to achieve a higher survival rate, anti-bacterial solutions were varied across 4 conditions.

All water used for the studies is distilled water that has been deionized by reverse osmosis. The Rosy Barb broodstock are kept in aqueous medium with a pH of 6.0 using SeaChem (SeaChem Laboratories Inc., Covington, Ga. 30014) Discus Buffer pH 6.0. The aqueous medium is also conditioned using Kent Black Water Expert (Kent Marine, Inc., Acworth, Ga. 30192) and a dechlorinator (Kordon Amquel) Kordon, div. of Novalek, Inc., 2242 Davis Ct., Hayward, Calif. 94545-1114. Each additive is 0.026% (or 264 μL/L). This solution is hereafter referred to as Blackwater. The Blackwater, along with the aqueous medium used for Zebrafish embryos, are the baseline aqueous media used as controls for the subsequent experiments described below. The Zebrafish are kept in aqueous medium at pH of 7.0 containing (0.01%) Methylene Blue, which is a concentration standard to Zebrafish larval rearing. The other three conditions described below are Blackwater+Nitrofurazone (B+N) water, Blackwater+Methylene Blue (B+M) water, and a Blackwater+Nitrofurazone+Methylene Blue (B+M+N) water. The B+N solution contains 60 mg of Nitrofurazone, 25 mg of Furazolidone, and 2 mg of Methylene Blue added per 5 gallons of Blackwater. These antibacterial agents are contained in the commercially available preparation termed, Furan-2 (Aquarium Pharmaceuticals, Inc., P.O. Box 218, Chalfont, Pa. 18914), which is added at a dilution of 0.5 capsules/5 gallons to achieve the appropriate concentrations of Nitrofurazone, Furazolidone and Methylene Blue. Therefore the w/v percentages of Nitrofurazone, Furazolidone and Methylene Blue are 0.157%, 0.066%, and 0.005%, respectively. The B+M solution has 1 mL of stock Methylene Blue Solution (1 mg Methylene blue/5 gallons water). The B+M+N solution contains a 50% dose of the two previous solutions. This leaves 5 conditions for the manipulated embryos to be kept in. A fertilized un-manipulated control group is kept in distilled water.

The embryos are microinjected, and placed in approximately equal amounts into each of five 100 mm×15 mm petri dishes (Fisher #08-757-12) containing 15 mL of aqueous medium. The embryos are maintained by cleaning out the dead eggs at 24 h and 48 h and adding fresh water twice a day. Then the number of larvae alive at hatch is recorded and they are screened for expression of the plasmid they were injected with.

Table 1 shows that survival rates at hatch were 42% for B, 30% for B+M, 41% for B+F, 39% for B+M+N, and 16% for Zebrafish water. 90% of the deaths occurred within 24 hours of fertilization. Out of these, the lowest survival rate (16%) was from the water used for Zebrafish, containing Methylene Blue and kept at a pH of 7.0. The other four water conditions held a survival rate of around 40% after 72 h. The two highest survival rates came from B water and B+N water at 42% and 41%, respectively. The B+M and B+M+N waters had lower survival rates of 30% and 39%, respectively.

TABLE 1
Survival and plasmid expression of Rosy Barb larvae
raised in five different water conditions.

| | | Number of | | Alive at | | Plasmid |
|---|---|---|---|---|---|---|
| Water | Number | Eggs Dead | | Hatch | Survival | Expression |
| Condition | Injected | 24 h | 48 h | 72 h | % | Positives |
| B | 74 | 41 | 2 | 31 | 42 | 6 |
| B + M | 80 | 51 | 5 | 24 | 30 | 3 |
| B + N | 76 | 41 | 4 | 31 | 41 | 13 |
| B + M + N | 62 | 33 | 5 | 24 | 39 | 5 |
| ZF | 67 | 47 | 9 | 11 | 16 | 0 |
| Control | 183 | 79 | 1 | 103 | 56 | n/a |

Methylene Blue has a negative effect on the Rosy Barb larvae. The effect of Methylene Blue can be seen by comparing the group with Methylene Blue, to the group that does not. The latter group has a higher survival rate. Out of the two water conditions having the higher survival rate (B and B+N), the rate of plasmid expression is significantly different. The B+N solution had over twice the amount of expressing larvae in its population. This is due to the effect of Furan-2 on the embryos. Out of the number of embryos injected, the plasmid only incorporates into a percentage of the embryos' genomes. Out of these, only a certain percentage can continue development without reaching a toxicity threshold and dying. The remaining incorporated embryos are most likely weakened compared to regularly developing embryos and thus guarded against bacteria by the Furan-2. Methylene Blue serves this purpose for Zebrafish but seems to be toxic, on some level, to Rosy Barbs. Therefore, water that contains Furan-2 instead of Methylene Blue, and has a pH and organic content appropriate to Rosy Barbs, will yield a higher survival and expression rate.

The protocol for tetraploid induction of Barbus conchonius (Rosy Barbs) is the same as Example 1 with the following changes: For Barbus conchonius, instead of distilled water in the 0.2 μL microcentrifuge tubes, pipette 100 μL of species specific conditioned water (hereinafter “Rosy Barb Water”). Related studies show that Rosy Barb water dramatically increases the viability of the Rosy Barb fertilized eggs. The Rosy Barb water contains 5 mL Kent Blackwater Expert (a organic concentrate used to simulate Amazonian waters) (Kent Marine, Inc., Acworth, Ga. 30192), ½ capsule Furan II (30 mg Nitrofurazone, 12.5 mg Furazolidone, 1 mg Methylene Blue), 1 teaspoon Seachem pH 6.0 Discus Buffer (SeaChem Laboratories Inc., Covington, Ga. 30014) and 5 mL Aquarium Pharmaceuticals, Inc. Stress Coat (as per manufacturer's directions, Aquarium Pharmaceuticals, Inc., P.O. Box 218, Chalfont, Pa. 18914; similar anti-stress preparations from other manufacturers will also work, such as ‘Bio-Coat by Marineland Labs, Moorepark, Calif. 93021) per 5 gallons of reverse osmosis water.

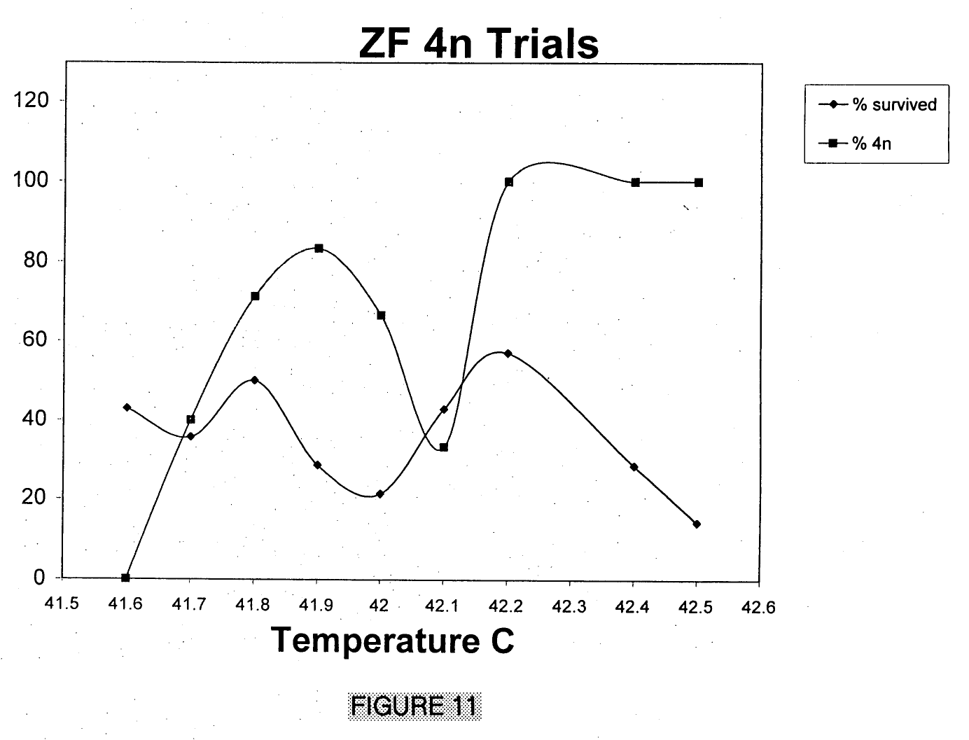

For Barbus conchonius, the same heat shock thermocycler protocol is used except the temperature of the first interval differs. For Barbus conchonius, the temperature of the first interval is approximately 40.6° C. instead of 41.9° C. As shown in FIG. 11, temperatures optimal for producing genome duplication in zebrafish do not yield surviving larvae in Barbus conchonius.

At the end of the heat shock protocol, open the 24 well culture cluster plate (Corning, Inc. item 3524) and obtain at least 200 mL of Rosy Barb water and an eyedropper. Open each tube, invert it, and squirt the Rosy Barb water up into the tube using the eyedropper to expel the embryos into a well in the 24 well culture cluster plate. Once all 24 wells are filled, incubate at room temperature (about 25° C.). Grow out the embryos and determine ploidy by doing FACS analysis on their fin clips.

For other fish the heat shock temperature in steps 1-5 must be optimized on an individual basis. Optimization is achieved by performing the heat shock procedure on multiple (e.g., 20) groups of eggs. Each group is shocked (step 1) at a different temperature. The reference temperature for step 1 that is examined should be based on the temperature at which the fish spawn in the wild. For example, neon tetras also spawn at room temperature (˜24° C.), and the gradient for step 1 would be examined between approximately 35° C. and 45° C., and the procedure for the other steps would be similar to that of Zebrafish or Rosy Barb.

### EXAMPLE 3

### Protocol for Salmon

Another example is salmon, which spawn at roughly ˜4° C. in the wild. Because this temperature is much lower than that of Rosy Barb, the temperature used for each of the steps will likely need to be about 20° C. lower than that used for Rosy Barb. Using Salmon as the example, the heat shock protocol for Step 1 would be performed at a temperature range from about 5° C. to 28° C. Following the heat shock, the temperature for Steps 2-5 would be close to approximately 4° C.

### EXAMPLE 4

### Protocol for Ploidy Analysis via Flow Cytometry

The effect of a timed heat shock on an embryo should be a duplication of the organism's genome. The method used to determine the success of this procedure is flow cytometry. Once a clutch of fish embryos has hatched, their cells dispersed and suspended in a solution containing DAPI (4′,6-diamidino-2-phenylindole, 10 μg/ml) to stain the DNA. The cell solution is filtered to remove clumps of cells, and run through a flow cytometer to measure the amount of DNA in each cell. Tetraploid cells (4n) should have twice as much DNA as a control diploid and thus have a value on the flow cytometer that is twice as high. Similarly, the octaploid cells (8n) should have twice as much DNA as tetraploid cells (4n). The protocol used by PiPets to determine the success of heat shocks to Zebrafish (Danio rerio) and Rosy barbs (Barbus conchonius) is as follows.

Upon the hatching of a heat-shocked clutch, the clutch is taken from the incubator to the flow cytometry workstation. Here the Partec Ploidy Analyser-PA I (Partec GmbH, Otto-Hahn-Straβe 32, D-48161 Munster, Germany) is turned on and allowed to warm up for ten minutes. Before counting experimental samples, the Analyser is calibrated according to the manufacturer's specifications. If the calibration is successful, a control run can be commenced using larva grown from eggs that have not been put through the genome duplication procedure.

Additional materials needed to prepare larva samples include double-edged razors (from any drug store), 100 mm×15 mm petri dishes (Fisher #08-757-12), CyStain UV (Partec #05-5001), 55×12 mm plastic test tubes, 50 μm filters (CellTrics #04-0041-2317), a Pasteur pipette, and a 1000 μL pipette with sterile tips. Transfer 3-5 control larvae into a petri dish with a Pasteur pipette, transferring as little water as possible. Aspirate the excess water without removing the larvae on the dish. Using a 1000 μL pipette, add approximately 100 μL of CyStain UV to the dish, covering the remaining larvae with the DAPI solution.

The larvae are then chopped by hand with a razor blade. Scores of small chops are necessary to sacrifice the entire group of larvae and release enough cells for the flow cytometer to have a successful run. Once this is completed, use the 1000 μL pipette to aspirate 500 μL of CyStain UV. Tilt the petri dish slightly to gather the liquid in one area and squirt the CyStain UV over the area that was chopped (visible markings on the petri dish and debris from chopping will be evident) and towards the gathered liquid in order to wash as many cells as possible into the solution. Aspirate the entire solution into the pipette tip and filter it using the 50 μm filter that is resting on top of the test tube. Follow this with an additional 500 μL of CyStain UV dispensed into the filter to wash the filter free of any remaining cells into the test tube, then bring the sample volume up to approximately 1.1 mL. After collecting any remaining fluid, discard the filter and place the tube on the machine, to start the FACS analysis.

Once the FACS analysis has commenced, an adjustment of the flow rate may be necessary depending on the density of the sample. A denser sample can run at a slower rate (0.25-0.50 μL/s) giving a counting rate of approximately 100 cells/s. A more dilute sample may need to run as high as (0.50-1.50 μL/s) to obtain a counting rat of 100 cells/s. Once an acceptable counting rate is established, the gain can be adjusted. When comparing diploid controls to possible tetraploids, it is advantageous to set the peak of the control to 100 on the x-axis. This will allow a tetraploid to register at 200 and make analysis simpler. The par gain for a control Rosy barb is set at 416 to achieve a peak at 100 on this instrument. The par gain is set at 385 for Zebrafish controls to register at 100 to normalize the data. Once the par gain is set for a certain species, it is not moved until the group of samples is completed. Counting is complete after approximately 6000 cells have been counted per run.

All references, including patents, patent applications, publications and any other material identified or described herein is hereby incorporated by reference in its entirety.

Having described the invention, many modifications thereto will become apparent to those skilled in the art to which it pertains without deviation from the spirit of the invention as defined by the scope of the appended claims.

1. A method of producing a marine organism having a triploid genome comprising breeding a first marine organism with a diploid genome to a second marine organism with a tetraploid genome to produce offspring having a triploid genome.

2. The marine organism produced by the method of claim 1 wherein said organism is incapable of breeding either with said transgenic organism or a non-transgenic organism and cannot produce viable or fertile transgenic progeny.

3. A method of producing a marine organism having a triploid genome comprising fertilizing tetraploid germ cells from a male or female organism, with diploid germ cells of the opposite sex to produce offspring having a triploid genome.

4. The marine organism produced by the method of claim 2 wherein said organism is incapable of breeding either with said transgenic organism or a non-transgenic organism and cannot produce viable or fertile transgenic progeny.

5. The marine organism of claim 2 wherein said marine organism is Brachydanio rerio.

6. The marine organism of claim 2 wherein said marine organism is Barbus conchonius.

7. The marine organism of claim 2 wherein said marine organism is Amphiprion ocellaris.

8. The marine organism of claim 2 wherein said marine organism is Premnas biaculeatus.

9. The marine organism of claim 2 wherein said marine organism is Amphiprion polymnus.

10. The marine organism of claim 2 wherein said marine organism is Amphiprion rubrocinctus.

11. The marine organism of claim 2 wherein said marine organism is Pseudochromis fridmani.

12. The marine organism of claim 2 wherein said marine organism is Pseudochromis sankeyi.

13. The marine organism of claim 2 wherein said marine organism is Pseudochromis springeri.

14. The marine organism of claim 2 wherein said marine organism is a Clam.

15. The marine organism of claim 2 wherein said marine organism is a Mussel.

16. The marine organism of claim 2 wherein said marine organism is an Oyster.

17. The marine organism of claim 2 wherein said marine organism is a Shrimp.

18. The marine organism of claim 2 wherein said marine organism is a Lobster.

19. A method of producing a marine organism having a hexaploid genome comprising breeding a first marine organism with a octaploid genome to a second marine organism with a tetraploid genome to produce offspring having a hexaploid genome.

20. The marine organism produced by the method of claim 19 wherein said organism is incapable of breeding either with said transgenic organism or a non-transgenic organism and cannot produce viable or fertile transgenic progeny.

21. A method of producing a marine organism having a hexaploid genome comprising fertilizing octaploid germ cells from a male or female organism, with tetraploid germ cells of the opposite sex to produce offspring having a hexaploid genome.

22. The marine organism produced by the method of claim 19 wherein said organism is incapable of breeding either with said transgenic organism or a non-transgenic organism and cannot produce viable or fertile transgenic progeny.

23. A method of making a triploid transgenic marine organism comprising having a first transgenic marine organism that is diploid and a second transgenic marine organism which is tetraploid and combining sperm and egg from said diploid and said tetraploid marine organism in such that the progeny resulting from said combination is triploid.

24. The triploid transgenic marine organism of claim 23 wherein said fish is incapable of breeding either with said transgenic marine organism or a non-transgenic marine organism and cannot produce viable or fertile transgenic progeny.

25. The transgenic marine organism of claim 23 wherein said marine organism is Brachydanio rerio.

26. The transgenic marine organism of claim 23 wherein said marine organism is Barbus conchonius.

27. The transgenic marine organism of claim 23 wherein said marine organism is Amphiprion ocellaris.

28. The transgenic marine organism of claim 23 wherein said marine organism is Premnas biaculeatus.

29. The transgenic marine organism of claim 23 wherein said marine organism is Amphiprion polymnus.

30. The transgenic marine organism of claim 23 wherein said marine organism is Amphiprion rubrocinctus.

31. The transgenic marine organism of claim 23 wherein said marine organism is Pseudochromis fridmani.

32. The transgenic marine organism of claim 23 wherein said marine organism is Pseudochromis sankeyi.

33. The transgenic marine organism of claim 23 wherein said marine organism is Pseudochromis springeri.

34. The marine organism of claim 23 wherein said marine organism is a Clam.

35. The marine organism of claim 23 wherein said marine organism is a Mussel.

36. The marine organism of claim 23 wherein said marine organism is an Oyster.

37. The marine organism of claim 23 wherein said marine organism is a Shrimp.

38. The marine organism of claim 23 wherein said marine organism is a Lobster.
